(19) United States
(12) Patent Application Publication
(10) Pub. No.: US 2010/0323374 A1
(43) Pub. Date: Dec. 23, 2010

(54) Serum Biomarkers for Diagnosing Liver Fibrosis and Method for Measuring the Same

(75) Inventors: Chun-Chia Cheng, Yonghe City (TW); Shui-Cheng Lee, Longtan Township (TW); Shiao-shiun Guan, Taoyuan City (TW)

Correspondence Address: Jackson Intellectual Property Group PLLC, 106 Starvale Lane, Shipman, VA, 22971, US

(73) Assignee: ATOMIC ENERGY COUNCIL-INSTITUTE OF NUCLEAR ENERGY RESEARCH, Taoyuan (TW)

(21) Appl. No.: 12/618,882

(22) Filed: Nov. 16, 2009

(30) Foreign Application Priority Data
Jun. 18, 2009 (TW) 098120397

(51) Int. Cl. G01N 33/53; G01N 33/92; C07K 14/00

(52) U.S. Cl. 435/7.92; 436/71; 530/350; 436/501

(57) ABSTRACT

Disclosed are serum biomarkers for diagnosing liver fibrosis and methods for measuring the same. The serum biomarkers obtained from human serum include alpha2-macroglobulin (“A2M”), vitamin D binding protein (“VDBP”), apolipoprotein AI (“ApoAI”). The methods involve with immunoassay using specific antibodies to detect the biomarkers, including enzyme-linked immunosorbent assay (“ELISA”), radio immune assay (“RIA”) and flexible multi-analyte profiling (“xMAP”). ELISA, RIA or xMAP is used to measure changes of protein concentration for the specific protein biomarkers in serum for diagnosing liver fibrosis with suffering hepatitis B or C or other liver diseases. The measurement is safe and accurate. The method can be used before and after treatment of liver fibrosis. Thus, it is possible to achieve early diagnosis and treatment advocated in the preventive medicine.

### FIELD OF THE INVENTION

The present invention relates to biomarkers for diagnosing liver fibrosis and methods for measuring the same, thus providing safe and accurate measurement of liver fibrosis in diagnosing or prognosing.

### DESCRIPTION OF THE RELATED ARTS

Liver fibrosis would lead to liver cirrhosis and some types of liver cancer, but liver fibrosis could be controlled or reversed with medicine. Therefore, early detection of liver fibrosis could be important to prevent the occurrence of liver cirrhosis or liver cancer.

As found in various documents, liver fibrosis is attributed to hepatitis, alcoholic hepatitis, drug intoxication, dystrophy, metabolic disorder or other diseases. Hepatitis B or C is caused by Hepatitis B or C virus. Alcoholic hepatitis is attributed to drinking overdose alcohol for a long period of time. Drug intoxication is attributed to liver toxic drug. In Taiwan, about 90% of patients with liver fibrosis suffer from hepatitis B, hepatitis C or alcoholic hepatitis. Besides, patients suffer from nonalcoholic steato-hepatitis (“NASH”) are increasing due to dystrophy or metabolic disease.

Currently, chronic hepatitis B, hepatitis C, non-alcoholic fatty liver disease (“NAFLD”), drug intoxication, alcoholic hepatitis or other hepatitis is diagnosed by HBsAg tests, anti-HCV tests, ultrasound, SGOT tests, SGPT tests or liver biopsy. Among them, liver biopsy is gold standard to determine the degree of liver fibrosis according to Metavir classification with five degrees, F0 to F4. The combination of liver biopsy with histology examination is used generally, but which has invasive character and sampling error. Hence, a lot of attention has been paid to search for noninvasive tests of liver fibrosis, e.g., routine biochemical tests, image tests and carbon-13 breath tests.

The routine biochemical tests cannot divide each stage of liver fibrosis although they can be used to tell sever liver fibrosis (or “liver cirrhosis”) from non-fibrosis. The image tests can be used to distinguish liver fibrosis F4 from F2, but it cannot be used to distinguish liver fibrosis F1 from F0. Furthermore, because of high intra-personal coefficient of variation (“CVᵢₙₜᵣₐ”) and complicated procedures, the carbon-13 breath tests cannot be used to provide reliable data for diagnosing liver fibrosis.

The tests of detecting protein biomarkers in serum are used directly to diagnose liver fibrosis, such as N terminal peptide of type III pro-collagen (“PIIINP”), tissue inhibitor of metalloproteinase 1 (“TIMP-1”), YKL-40, hyaluronic acid, gamma glutamyl transferase (“GGT”), haptoglobin or total bilirubin. However, their reliabilities are not good enough. In addition to the foregoing problem, the changes of fibrotic formation in other organs would interfere with the accuracy of detecting process while using those serum biomarkers.

The present invention is therefore intended to obviate or at least alleviate the problems encountered in prior art.

### SUMMARY OF THE INVENTION

It is the primary objective of the present invention to provide safe and accurate measurement of liver fibrosis in diagnosing or prognosing by providing serum biomarkers for diagnosing liver fibrosis and methods for measuring the same.

To achieve the foregoing objective, a set of protein biomarkers are used in an immunoassay technique for example. The protein biomarkers are multiply variable proteins related to liver fibrosis selected from human serum. The set of protein biomarkers includes α-2 macroglobulin with molecular weight of 162072, vitamin D binding protein with molecular weight of 52780 and apolipoprotein AI with molecular weight of 28061.

Other objectives, advantages and features of the present invention will become apparent from the following description referring to the attached drawings.

### BRIEF DESCRIPTIONS OF THE DRAWINGS

The present invention will be described via detailed illustration of the preferred embodiment referring to the drawings.

FIG. 1 is a flow chart of a method for obtaining protein biomarkers according to the preferred embodiment of the present invention.

FIG. 2 is a flow chart of a method for diagnosing liver fibrosis using the biomarkers obtained in the method shown in FIG. 1.

FIG. 3 is a chart of the relation between liver fibrosis and the concentration of A2M obtained in the method shown in FIG. 1.

FIG. 4 is a chart of the relation between liver fibrosis and the concentration of VDBP obtained in the method shown in FIG. 1.

FIG. 5 is a chart of the relation between liver fibrosis and the concentration of ApoAI obtained in the method shown in FIG. 1.

FIG. 6 is a table of the concentrations of the protein biomarkers versus the degrees of liver fibrosis.

### DESCRIPTION OF THE PREFERRED EMBODIMENT

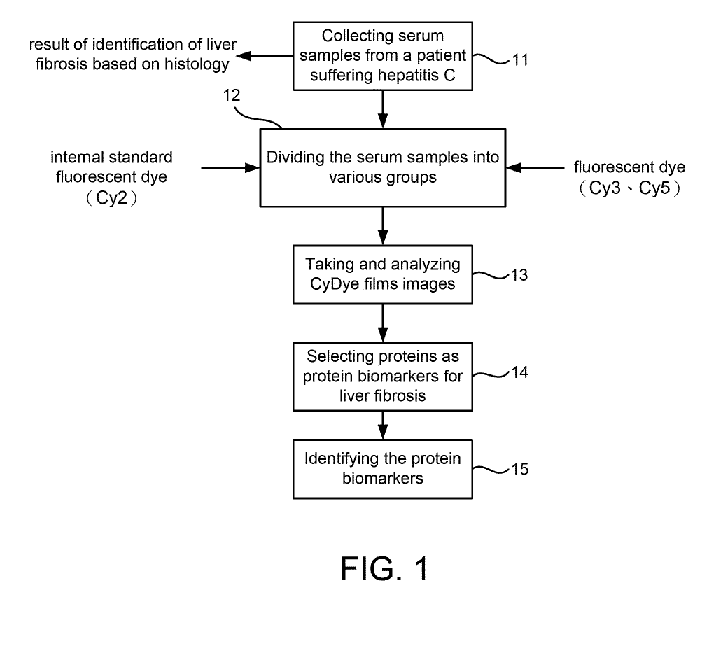

Referring to FIG. 1, there is shown a method for obtaining protein biomarkers for diagnosing liver fibrosis according to the preferred embodiment of the present invention. The method includes the steps of obtaining serum samples, 2-dimensional differential gel electrophoresis (“2D-DIGE”), taking and analyzing CyDye films, selecting possible protein biomarkers for liver fibrosis and identifying the selected protein biomarkers. At 11, serum samples are collected from patients suffering hepatitis C virus. Tissue samples are also taken from the liver of the patients by liver biopsy. The tissue samples are classified based on histology according to METAVIR classification. The result is taken a gold standard.

At 12, the serum samples are divided into various groups. Different amounts of fluorescent dyes are provided on various films regarding the serum samples. One of the fluorescent dyes is taken as an internal standard for the 2D-DIGE.

At 13, the images on the films marked with the fluorescent dyes are overlapped. Differential in-gel analysis (“DIA”) and biological variation analysis (“BVA”) are conducted. By modular calculation, a T-test value and a one-way ANOVA value are used to select the putative biomarkers.

At 14, the reliability of each of the proteins is determined based on the T-test value and the one-way ANOVA value. Three of the proteins are selected as the protein biomarkers for liver fibrosis.

At 15, the films of the selected protein biomarkers are dyed. A spectrograph and a MASCOT protein identification station are used to analyze and identify the selected protein biomarkers.

The selected protein biomarkers are multiply variable proteins related to liver fibrosis. The selected protein biomarkers are α-2 macroglobulin (“A2M”), vitamin D binding protein (“VDBP”) and apolipoprotein AI (“ApoAI”) for example. The selected protein biomarkers can be used to diagnose liver fibrosis.

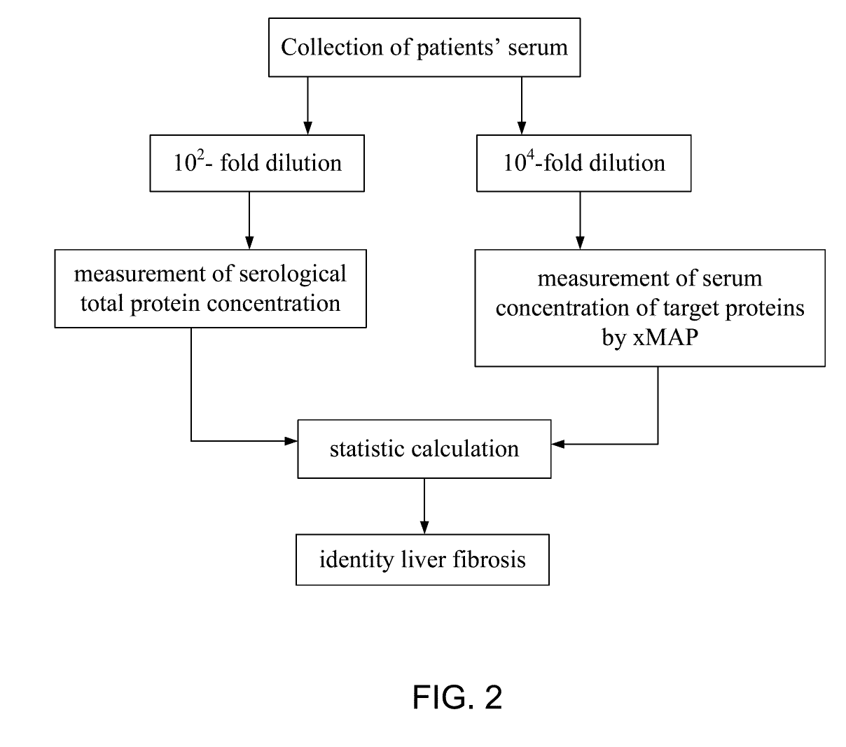

Referring to FIG. 2, there is shown a method for diagnosing liver fibrosis using the biomarkers selected in the method shown in FIG. 1. Based on current immunity diagnose techniques, the concentration of at least one of the selected protein biomarkers in serum is measured for diagnosing liver fibrosis.

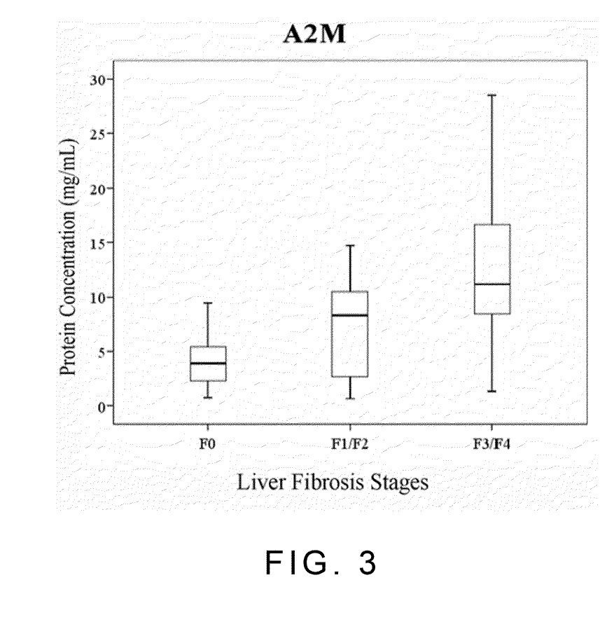

Referring to FIG. 3, there is shown the relation between liver fibrosis and the concentration of A2M obtained in the method shown in FIG. 1. The concentration of A2M of the patients is higher than that of healthy people. The concentration of A2M of the patients is 7.24±4.29 mg/ml at F1/F2 and 12.97±6.79 mg/ml at F3/F4 much higher than the normal value of 4.31±2.78 mg/ml (p<0.01).

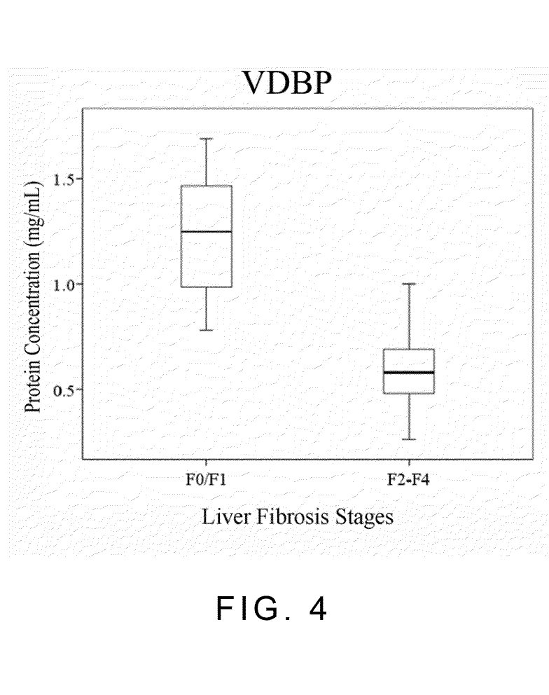

Referring to FIG. 4, there is shown the relation between liver fibrosis and the concentration of VDBP obtained in the method shown in FIG. 1. The concentration of VDBP of the patients is lower than that of a healthy people. The concentration of VDBP of the patients is 1.22±0.29 mg/ml at F0/F1 and 0.60±0.18 mg/ml (p<0.01) at F2-F4.

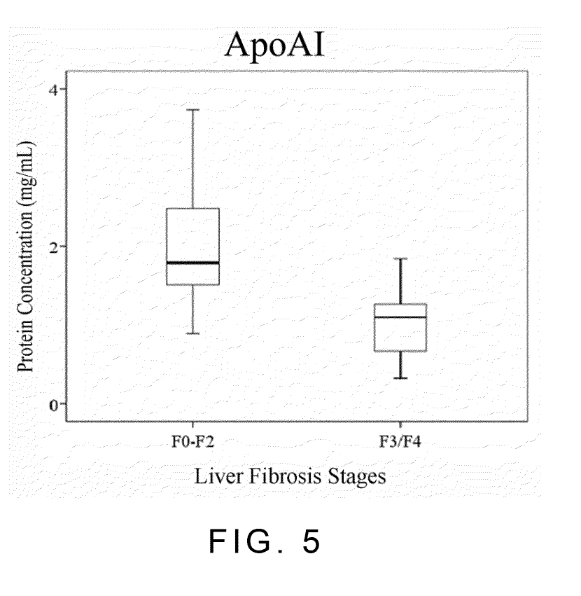

Referring to FIG. 5, there is shown the relation between liver fibrosis and the concentration of ApoAI obtained in the method shown in FIG. 1. The concentration of ApoAI of the patients is lower than that of a healthy people. The concentration of ApoAI of the patients is 1.98±0.71 mg/ml at F0 to F2 and 1.06±0.45 mg/ml at F3/F4 much lower than the normal value of 4.31±2.78 mg/ml (p<0.01).

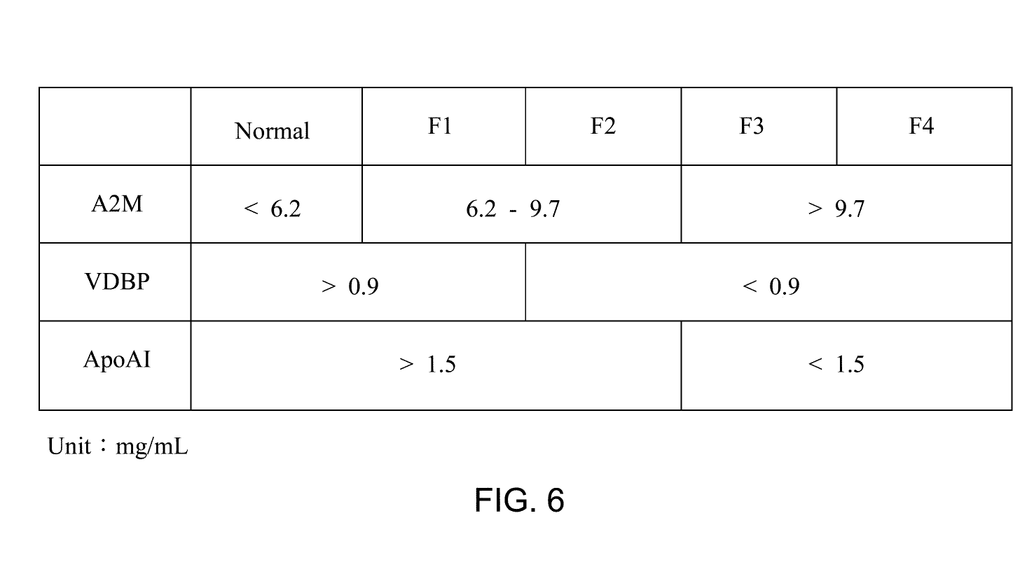

Referring to FIG. 6, there is shown a table of the concentrations of the protein biomarkers versus the degrees of liver fibrosis. At F0, A2M is lower than 6.2 mg/ml, VDBP is higher than 0.2 mg/ml, and ApoAI is higher than 1.5 mg/ml. At F1, A2M is 6.2 to 9.7 mg/ml, VDBP is higher than 0.9 mg/ml, and ApoAI is higher than 1.5 mg/ml. At F2, A2M is 6.2 to 9.7 mg/ml, VDBP is lower than 0.9 mg/ml, and ApoAI is higher than 1.5 mg/ml. At F3/F4, A2M is higher than 9.7 mg/ml, VDBP is lower than 0.9 mg/ml, and ApoAI is lower than 1.5 mg/ml.

More details of the method will be given as follows:

First Step

The blood samples are taken from the patient and put still of 15 minutes. Then, supernatant liquid is taken by centrifugal, and reserved in a refrigerator at −80 degrees Celsius.

Second Step

10 ug of antibodies is disposed in PBS buffer (pH 7.4) for dialysis overnight.

Then, 10 ug of 50 mg/ml 3-dimethylaminopropyl carbodiimide hydrochloride (“EDC”) solution and N-hydroxysuccinimide (“S-NHS”) solution are provided. The EDC and the S-NHS are added into 100 ul of microspheres sequentially. Reaction lasts for 20 minutes before the solution is subjected to a centrifugal force of 14000 g for 4 minutes. Then, supernatant liquid is removed from the solution and dissolved in 100 ul of PBS buffer.

The antibodies are mixed with the microspheres for 2 hours before the solution is subjected to a centrifugal force 14000 g for 4 minutes. Then, supernatant liquid is removed from the solution and dissolved in 250 ul of buffer. Reaction lasts of 30 minutes before the solution is subjected to a centrifugal force or 14000 g for 4 minutes. Then, the solution is dissolved in 150 ul of storing solution.

Reaction with anti-rabbit antibody conjugated biotin reaction is conducted for 30 minutes. Reaction with streptavidin conjugated phycoerythrin (“SA-PE”) is conducted for 10 minutes. Measured with BioPlex200, the florescent intensity must be higher than 2000 FL.

Third Step

The serum samples are diluted by 1×10² and 1×10⁴, respectively. The concentrations of all of the proteins, A2M, VDBP and ApoAI in the serum are measured.

Fourth Step

After the measurement, the concentrations of A2M, VDBP and ApoAI in the serum are known. At F0, A2M is lower than 6.2 mg/ml, VDBP is higher than 0.2 mg/ml, and ApoAI is higher than 1.5 mg/ml. At F1, A2M is 6.2 to 9.7 mg/ml, VDBP is higher than 0.9 mg/ml, and ApoAI is higher than 1.5 mg/ml. At F2, A2M is 6.2 to 9.7 mg/ml, VDBP is lower than 0.9 mg/ml, and ApoAI is higher than 1.5 mg/ml. At F3/F4, A2M is higher than 9.7 mg/ml, VDBP is lower than 0.9 mg/ml, and ApoAI is lower than 1.5 mg/ml.

Fifth Step

A2M, VDBP and ApoAI can be used in other immunoassay techniques such as RIA and ELISA. By measuring the concentrations of A2M, VDBP and ApoAI, liver fibrosis is diagnosed.

Advantageously, the concentration of at least one of three protein biomarkers in serum of a patient suffering hepatitis B or C or other liver diseases is measured to diagnose liver fibrosis according current immunoassay techniques. The protein biomarkers are multiply variable proteins related to liver fibrosis. Therefore, the measurement of the degree of liver fibrosis in diagnosing before treatment or in tracing after treatment is safe and accurate.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

What is claimed is:

1. A method for determining the degree of liver fibrosis by using a set of protein biomarkers in an immunoassay technique, wherein the protein biomarkers are multiply variable proteins related to liver fibrosis selected from human serum, the set of protein biomarkers comprises:
α-2 macroglobulin with molecular weight of 162072;
vitamin D binding protein with molecular weight of 52780; and
apolipoprotein AI with molecular weight of 28061.

2. A protein biomarker for use in the method according to claim 1.

3. A method for measuring a protein biomarker for diagnosing liver fibrosis comprising the steps of:
collecting serum samples from a patient;
reacting the serum samples with protein biomarker antibodies; and
testing the solution and determining the degree of liver fibrosis.

4. The method according to claim 3 comprising the step of using an instrument selected from a group consisting of xMAP, ELISA and RIA.
